(19) United States
(12) Patent Application Publication
(10) Pub. No.: US 2008/0172055 A1
(43) Pub. Date: Jul. 17, 2008

(54) Method for indicating the position and orientation of a surgical tool and apparatus for performing this method

(75) Inventors: Benoit Mollard, Echirolles (FR); Cyril Perot, La Tronche (FR); Sergej Kammerzell, Engen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/827,096

(22) Filed: Jul. 9, 2007

Related U.S. Application Data
(63) Continuation of application No. PCT/EP2005/014039, filed on Dec. 24, 2005.

(30) Foreign Application Priority Data
Jan. 17, 2005 (DE) 10 2005 003 318.0

(51) Int. Cl. A61F 5/00; A61B 17/58; A61B 17/56

(52) U.S. Cl. 606/87; 606/102; 606/53

(57) ABSTRACT

In a method for indicating the position and orientation of a tool for preparation of a femur for a femur implant which with a joint ball fits into the acetabulum of the pelvis or into a joint socket inserted in the pelvis or for preparation of such a femur implant for insertion into a femur relative to the femur and to the center point of the joint ball, in order to provide the operating surgeon with an easily understandable representation for targeted positioning of the tool or the femur implant, it is proposed that the center point of the joint ball and a line of intersection of a transversal plane of the femur and an instrument plane, which line of intersection passes through the center point of the joint ball, be represented in this transversal plane of the femur at the level of the center point of the joint ball.

This application is a continuation of international application number PCT/EP2005/014039 filed on Dec. 24, 2005.

The present disclosure relates to the subject matter disclosed in international application number PCT/EP2005/014039 of Dec. 24, 2005 and German application number 10 2005 003 318.0 of Jan. 17, 2005, which are incorporated herein by reference in their entirety and for all purposes.

### BACKGROUND OF THE INVENTION

The invention relates to a method for indicating the position and orientation of a tool for preparation of a femur for a femur implant which with a joint ball fits into the acetabulum of the pelvis or into a joint socket inserted in the pelvis or for preparation of such a femur implant for insertion into a femur relative to the femur and to the center point of the joint ball.

In hip joint operations, it is necessary to insert a femur implant into the femur after the head of the femur has been removed. Normally, the femur implant is adapted to the natural shape of the bone and has a shaft introducible into the femur, a femoral neck extending away from the shaft at an angle thereto, and on the femoral neck a joint ball which fits into the joint socket of the pelvis, either into the natural joint socket or, in most cases, into an artificial joint socket.

The femur must be prepared for insertion of the femur implant into it, and it is standard practice to do this with rasp-like tools which prepare the medullary space of the femur in such a way that the shaft of the femur implant can be inserted with a precise fit into the femur, i.e., in such a way that the implant assumes the desired position and orientation relative to the femur, so that the femur is correctly positioned as a result of engagement of the joint ball of the femur implant in the acetabulum.

The operating surgeon must guide the rasp-like tool correctly or, when inserting the femur implant, position it itself correctly relative to the femur, and, to do so, it is known to indicate to the operating surgeon on a display screen which angles the rasp-like tool or the femur implant assume relative to the femur. This does enable the operation to be performed, but the indication of numerical angular data is not readily understandable and first has to be converted into the necessary manipulations by the operating surgeon.

An object of the invention is to so develop an indicating method of the kind described at the outset that the change in position and orientation, i.e., the position of the tool preparing the femur or of the femur implant, is shown so clearly to the operating surgeon that without any further consideration, by changing the position, he can reach the desired position and also read it off from a display.

### SUMMARY OF THE INVENTION

This object is accomplished in accordance with the invention in a method of the kind described at the outset in that the center point of the joint ball and a line of intersection of a transversal plane of the femur and an instrument plane, which line of intersection passes through the center point of the joint ball, are represented in this transversal plane of the femur at the level of the center point of the joint ball.

Therefore, on the display screen the operating surgeon can see the center point of the joint ball, on the one hand, and the line of intersection, on the other hand, which indicates to him at what angle in the transversal plane the instrument plane intersects the transversal plane. The instrument plane may be a plane of a surgical, rasp-like instrument, but the instrument plane may also be a plane of the femur implant.

For example, it may be provided that the instrument plane is selected so that it is the center plane of the tool. The shafts and then also the corresponding rasp-like tools often have an oval or elongate cross section, and the long axis points in the direction of the femoral neck of the femur prosthesis, i.e., in the direction towards the joint ball. If this longitudinal center plane of the shaft is used as instrument plane, it is ensured that this plane will pass through the joint ball and hence indicate to the operating surgeon how the direction runs from the shaft to the center point of the joint in the transversal plane.

In another preferred embodiment, it may be provided that the instrument plane is selected so that the projection of the longitudinal axis of a holding arm connecting the shaft of a femur implant or of a tool to the joint ball onto the transversal plane lies in the instrument plane. It is then not the center plane of the tool or instrument that is used as instrument plane, but a plane that passes through this projection. In this case, too, it is ensured that the instrument plane passes through the joint ball.

In order to make the function of the center point of the joint clear to the operating surgeon, it may be provided, in accordance with a preferred embodiment, that an image of a transversal section of the pelvis at the level of the center point of the acetabulum receiving the joint ball is copied into the representation of the transversal plane of the femur and of the line of intersection such that the center point of the joint ball and the center point of the acetabulum coincide. The transversal plane is then a plane which passes through the two center points of the acetabula and extends horizontally in a patient standing upright. This may be a cross-sectional view, but also a plan view of the pelvis, in which the position of the center point of the acetabulum is made visible by the pelvis being partly represented transparently. It is only essential that it be indicated symbolically to the operating surgeon by this representation that the center point of the joint of the femur is located in the acetabulum of the pelvis.

To locate the transversal plane of the femur, the position and orientation of the center point of the joint ball and of the knee joint may preferably be determined, for example, by palpation of the kneecap, and, in addition, the mechanical femur axis as line connecting the two points, and the transversal plane then extends perpendicularly to this mechanical femur axis and passes through the center point of the joint ball.

Upon changing the position and orientation of the tool and/or the femur implant, the operating surgeon can immediately see in the described representation how the line of intersection moves between instrument plane and transversal plane of the femur; upon turning the shaft of the machining tool or of the femur implant, the angle of this line of intersection will change in the representation; upon lateral displacement of the tool shaft or of the implant shaft without turning, the line will move parallel to itself. A correct position and orientation of the tool shaft and of the femur implant shaft is obtained when the line of intersection passes through the image of the center point of the joint ball. As the instrument plane itself passes through the center point of the joint ball, this indicates to the operating surgeon that both the angular orientation and the lateral displacement of the tool shaft or of the femur implant shaft are chosen correctly.

A further object of the invention is to create a device for performing this method.

This object is accomplished, in accordance with the invention, by a device comprising a navigation system, marking elements associated with this navigation system for the femur, for the pelvis and for the tool or the femur implant, furthermore, by a data processing installation and a display activated by this data processing installation, it being provided that the data processing installation is programmed to represent on the display the center point of the joint ball and a line of intersection of a transversal plane of the femur and an instrument plane, which line of intersection passes through the center point of the joint ball, in this transversal plane of the femur at the level of the center point of the joint ball.

It is advantageous for the data processing installation to be programmed to select the center plane of the tool as instrument plane.

In another embodiment, the data processing installation may be programmed to select the instrument plane such that the projection of the longitudinal axis of a holding arm connecting the shaft of a femur implant or of a tool to the joint ball onto the transversal plane of the femur lies in the instrument plane.

It is particularly expedient for the data processing installation to be programmed to copy an image of a transversal section of the pelvis at the level of the center point of the acetabulum receiving the joint ball into the representation of the transversal plane of the femur and of the line of intersection such that the center point of the joint ball and the center point of the acetabulum coincide.

In order to locate the transversal plane of the femur, the data processing installation may be further programmed to determine from the position and orientation of the center point of the joint ball and of the knee joint a mechanical femur axis as line connecting the two points, and to calculate the transversal plane as extending perpendicularly to this mechanical femur axis and passing through the center point of the joint ball.

The following description of preferred embodiments of the invention serves in conjunction with the drawings for further explanation.

### BRIEF DESCRIPTION OF THE DRAWINGS

FIG. 1: shows a schematic view of the pelvis and the femur of a patient and a navigation system with a data processing installation and a display device;

FIG. 2: shows a schematic view of the pelvis and part of the femur with an inserted and navigated machining tool, in which the femoral transversal plane and the instrument plane are indicated;

FIG. 3: shows a view of the display indicating the line of intersection of the femoral transversal plane and the instrument plane, the center point of the joint head and a copied-in representation of the pelvis;

FIG. 4: shows a cross-sectional representation of the shaft of a rasp-like machining tool in accordance with line 4-4 in FIG. 2;

FIG. 5: shows a schematic side view of a femur implant with a shaft, a femoral neck and a joint ball; and

FIG. 6: shows a sectional view of the femur implant of FIG. 5 taken along line 6-6 in FIG. 5.

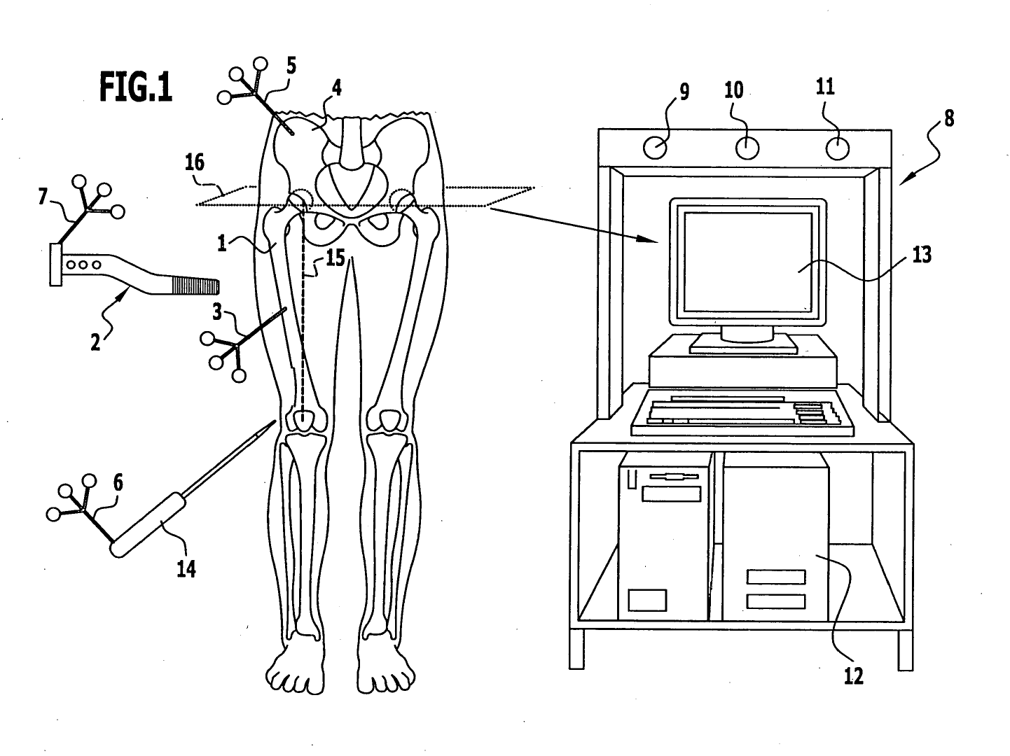

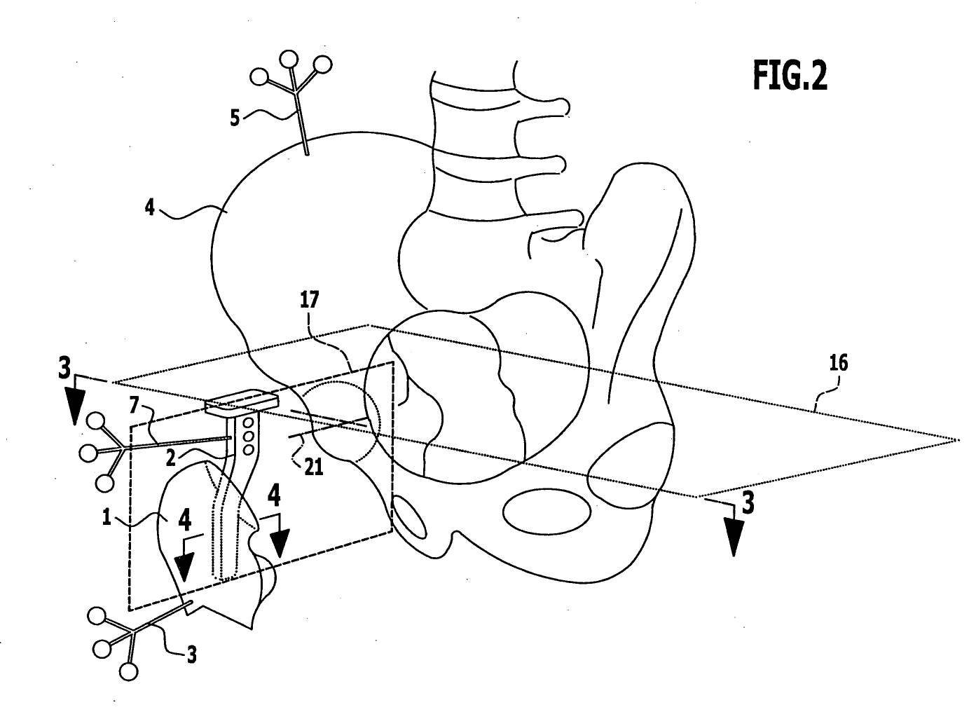

### DETAILED DESCRIPTION OF THE INVENTION

The method according to the invention is used to prepare the femur 1 of a patient for receiving a femur implant or to insert a femur implant into the femur. This is necessary in a hip joint operation during which the natural hip joint is replaced by an artificial joint socket in the pelvis and a femur implant with a joint ball which, after removal of the natural femur head, is inserted, in order to replace this, into the remaining femur. The femur implants normally have a shaft which is driven into the medullary space of the femur, for which this medullary space must be appropriately prepared. This preparation is carried out with the aid of a rasp-like tool 2, which is constructed very similarly to the shaft of the femur implant and shapes the medullary space of the femur 1 appropriately, so that on introducing the shaft of the femur implant, the positioning of the femur implant in the femur is determined by the geometry of the prepared medullary cavity. It is, therefore, very important that when preparing the femur, the tool 2 be driven into the femur in precisely the desired angular orientation and lateral position. It is, of course, also essential that the femur implant be inserted into the femur in the correct position. Here correct position means that the laterally protruding arm of the femur component, which usually carries a joint ball at its end, assumes the correct orientation relative to the pelvis and to the joint socket in the pelvis.

As a result of preparation for the operation, the operating surgeon knows where to place the joint head relative to the natural femur. Also, the geometrical data of the femur implant are known, so that when inserting the femur implant into the femur it is at any rate ascertainable which position of the femur implant in the femur results in which position of the joint head.

In order to check this, there are permanently and rigidly attached to the femur 1 a marking element 3, to the pelvis 4 a marking element 5, and to the tool 2 a marking element 7. The tool 2 could be replaced in like manner by the femur implant. Therefore, solely the tool 2 will be referred to in the following.

The position of the marking elements 3, 5 and 7 can be determined by a navigation system 8, known per se, which, for example, comprises three transmitters 9, 10, 11, spatially separate from one another, which emit infrared radiation which is reflected by spatially separate reflectors of the marking elements 3, 5, 7 and is picked up again by the transmitters 9, 10, 11 which also operate as receivers. Both the orientation and the position of the marking elements in space can be calculated from the radiation, i.e., the position of each individual marking element. Since the marking elements are rigidly connected to the femur, the pelvis and the tool, respectively, the respective position of the femur, the pelvis and the tool in space can, therefore, also be determined at any time.

The corresponding sets of positional data are fed by the navigation system 8 to a data processing installation 12, which uses these data to calculate further data and, inter alia, can generate an optical display on a display screen 13.

The position of the center point of the joint ball relative to the femur is first determined using the described device. This determination is carried out before removal of the natural femur head simply by the femur being swivelled relative to the pelvis. During this swivel movement, the marking element 3 of the femur 1 moves on a ball cup whose center point is the center point of the joint head. From this movement and the corresponding sets of positional data the data processing installation can determine the center point of the joint ball relative to the marking element 3 and hence relative to the femur 1.

The position of the knee joint is also determined. This can be carried out by palpation using a palpating element 14, which has a marking element 6 and is placed with its tip, for example, on the kneecap of the patient. From the positional data of the navigated palpating element 14 and the geometrical data thereof the data processing installation can calculate the exact position of the palpated marking point on the kneecap, which is used as knee joint point. The data processing installation then forms the line connecting the center point of the joint head and of the knee joint. This connecting line is denoted as mechanical femur axis 15.

From this mechanical femur axis 15 and the position of the center point of the joint ball, the data processing installation 12 further calculates a femoral transversal plane 16 extending perpendicularly to the mechanical femur axis 15 and passing through the center point of the joint. This is a section plane which is associated with the femur.

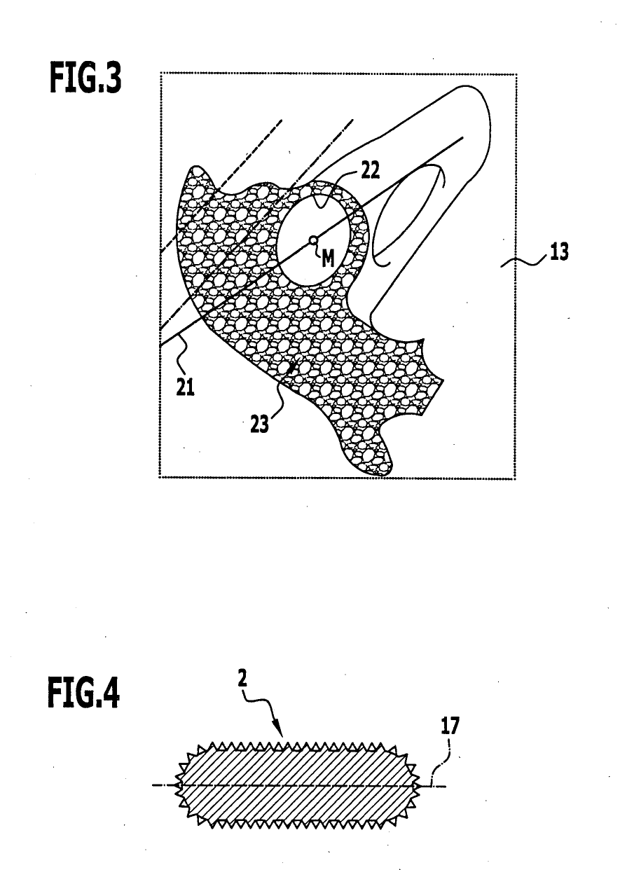

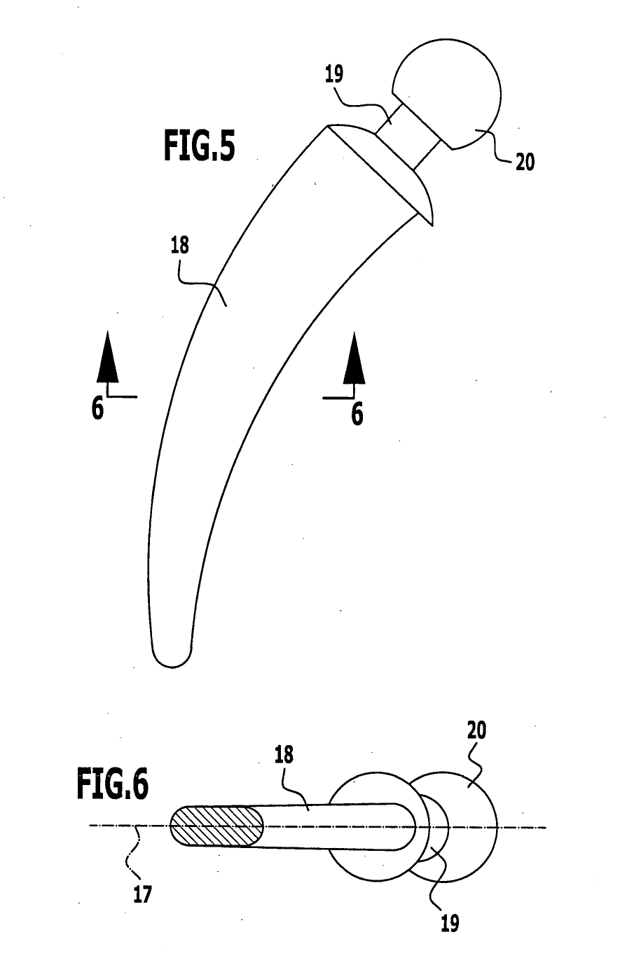

An instrument plane 17 is associated with the tool 2. This can be, for example, a longitudinal center plane. As will be apparent from FIG. 4, the shaft is, for example, of oval construction with a long axis and a short axis, and the longitudinal center plane extends along the long axis and in the longitudinal direction of the shaft.

If a femur implant with a similar shaft shape is used, it may be provided that the femoral neck 19 adjoining the shaft 18, which carries the joint ball 20, likewise extends in the longitudinal center plane of the femur implant, as depicted in FIG. 6. It may, however, also be provided that this connecting arm forms an angle with this plane. During insertion of the shaft in conformance with the shape into an opening prepared by the tool, the femoral neck does then not lie in the longitudinal center plane of the instrument, but in the plane opened up by the connecting arm between shaft and joint ball, and this forms the instrument plane used by the data processing installation.

The data processing installation respectively receives from the navigation system the positional data of the tool 2 and calculates therefrom the position of the respective instrument plane and also the line of intersection 21 of the instrument plane 17 and the femoral transversal plane 16. On the display, this line of intersection is indicated in the femoral transversal plane 16, and also the center point M of the joint ball 20.

If this line of intersection 21 passes through the center point M of the joint ball 20, the tool 2 or the femur implant is correctly orientated, for it is then ensured that the instrument plane will pass through the center point of the joint ball. If, on the other hand, there are deviations, the operating surgeon knows that he has to move sideways or turn the tool or the femur implant in order to attain the desired position.

In addition, a plan view or a horizontal cross-sectional view of a pelvis is depicted on the display screen 13. This pelvis representation 23 can be schematic and does not require the anatomical data of the patient. It merely serves to indicate to the operating surgeon that the center point M is simultaneously also the center point of the joint socket receiving the joint ball. For this purpose, this joint socket 22 is visible in the sectional representation or in a transparent representation. The pelvis representation 23 is so arranged that the center point of the joint socket coincides with the center point M of the joint ball 20.

Normally the femoral transversal plane will not coincide with the section plane resulting from a horizontal cross-sectional view of the pelvis extending through the hip joint center points.

However, it is possible to determine from the position of the femoral transversal plane relative to this horizontal cross-sectional plane a set of data which describes how the femur and the parts connected to the femur have to be moved and swivelled in order that the femoral transversal plane will coincide with this horizontal cross-sectional plane which is denoted as transversal plane of the pelvis. When the femur is in a position in which these two planes coincide, one refers to a neutral position of the femur.

Using this set of transformation data it would then also be possible to represent a section in the transversal plane of the pelvis instead of a section in the transversal plane of the femur, as both planes coincide in the neutral position. However, a precondition is that the positional and orientation data indicating the position of the femur and the parts rigidly connected to it be so converted that the femur is in the neutral position relative to the pelvis.

1. Method for indicating position and orientation of a tool for preparation of a femur for an implant which with a joint ball fits into an acetabulum of a pelvis or into a joint socket inserted in the pelvis or for indicating position and orientation of the femur implant for insertion into a femur relative to the femur and to a center point of the joint ball, comprising:
determining a transversal plane of the femur;
determining the center point of the joint ball;
determining an instrument plane for one of the tool or the femur implant;
determining a line of intersection of the transversal plane of the femur and the instrument plane;
displaying a representation of the center point of the joint ball and the line of intersection on a display, the line of intersection being represented in the transversal plane of the femur at a level of the center point of the joint ball.

2. Method in accordance with claim 1, wherein the instrument plane is selected to be a center plane of one of the tool or the femur implant.

3. Method in accordance with claim 1, wherein the instrument plane is so selected that a projection of a longitudinal axis of a holding arm connecting a shaft of one of the femur implant or the tool to the joint ball onto the transversal plane lies in the instrument plane.

4. Method in accordance with claim 1, further comprising:
providing an image of a transversal section of the pelvis at a level of the center point of an acetabulum receiving the joint ball in the representation such that the center point of the joint ball and the center point of the acetabulum coincide.

5. Method in accordance with claim 1, wherein determining the transversal plane of the femur comprises:
determining a position and orientation of the center point of the joint ball and of a center point of a knee joint, and
determining a mechanical femur axis as a line connecting the center points,
wherein the transversal plane of the femur extends perpendicularly to the mechanical femur axis and passes through the center point of the joint ball.

6. Method in accordance with claim 1, further comprising:
determining correct orientation of one of the tool or the femur implant relative to the femur when the line of intersection passes through the center point of the joint ball.

7. Method in accordance with claim 4, wherein determining the transversal plane of the femur comprises:
determining a position and orientation of the center point of the joint ball and of a center point of a knee joint, and
determining a mechanical femur axis as a line connecting the center points,
wherein the transversal plane of the femur extends perpendicularly to the mechanical femur axis and passes through the center point of the joint ball.

8. Method in accordance with claim 4, further comprising:
determining correct orientation of one of the tool or the femur implant relative to the femur when the line of intersection passes through the center point of the joint ball.

9. Method in accordance with claim 5, further comprising:
determining correct orientation of one of the tool or the femur implant relative to the femur when the line of intersection passes through the center point of the joint ball.

10. Apparatus for indicating position and orientation of a tool for preparation of a femur for an implant which with a joint ball fits into an acetabulum of a pelvis or into a joint socket inserted in the pelvis or for indicating position and orientation of the femur implant for insertion into a femur relative to the femur and to a center point of the joint ball, comprising:
a navigation system with marking elements associated with the femur, the pelvis and one of the tool or the femur implant for determining a corresponding position of the tool or the femur implant, and
a data processing installation associated with the navigation system for activating a display and for determining a transversal plane of the femur, the center point of the joint ball, an instrument plane for one of the tool or the femur implant, and a line of intersection of the transversal plane of the femur and the instrument plane,
wherein the data processing installation is programmed to represent on the display the center point of the joint ball and the line of intersection, the line of intersection being represented in the transversal plane of the femur at a level of the center point of the joint ball.

11. Apparatus in accordance with claim 10, wherein the data processing installation is programmed to select the center plane of one of the tool or the femur implant as the instrument plane.

12. Apparatus in accordance with claim 10, wherein the data processing installation is programmed to select the instrument plane such that a projection of a longitudinal axis of a holding arm connecting a shaft of one of the femur implant or the tool to the joint ball onto the transversal plane of the femur lies in the instrument plane.

13. Apparatus in accordance with claim 10, wherein the data processing installation is programmed to provide in the representation an image of a transversal section of the pelvis at a level of the center point of an acetabulum receiving the joint ball such that the center point of the joint ball and the center point of the acetabulum coincide.

14. Apparatus in accordance with claim 10, wherein in order to locate the transversal plane of the femur, the data processing installation is programmed to:
determine a position and orientation of the center point of the joint ball and of a center point of a knee joint,
determine a mechanical femur axis as a line connecting the center points, and
wherein the transversal plane of the femur extends perpendicularly to the mechanical femur axis and passes through the center point of the joint ball.

15. Apparatus in accordance with claim 13, wherein in order to locate the transversal plane of the femur, the data processing installation is programmed to:
determine a position and orientation of the center point of the joint ball and of a center point of a knee joint,
determine a mechanical femur axis as a line connecting the center points, and
wherein the transversal plane of the femur extends perpendicularly to the mechanical femur axis and passes through the center point of the joint ball.

16. Apparatus in accordance with claim 10, wherein a correct orientation of one of the tool or the femur implant relative to the femur is determined when the line of intersection passes through the center point of the joint ball.
